(19) United States
(12) Patent Application Publication
(10) Pub. No.: US 2014/0194879 A1
(43) Pub. Date: Jul. 10, 2014

(54) GLENOID AUGMENT PREPARATION INSTRUMENT

(71) Applicants: Cleveland Clinic Foundation, Cleveland, OH (US); COORSTEK MEDICAL LLC d/b/a IMDS, Providence, UT (US)

(72) Inventor: Dinesh Koka, Orlando, FL (US)

(73) Assignees: Cleveland Clinic Foundation, Cleveland, OH (US); COORSTEK MEDICAL LLC d/b/a IMDS, Providence, UT (US)

(21) Appl. No.: 14/206,103

(22) Filed: Mar. 12, 2014

Related U.S. Application Data
(63) Continuation-in-part of application No. 13/708,799, filed on Dec. 7, 2012.
(60) Provisional application No. 61/777,538, filed on Mar. 12, 2013.

(51) Int. Cl. A61B 17/16

(52) U.S. Cl. CPC A61B 17/16
USPC 606/79

(57) ABSTRACT

A system for preparing a bone for receiving an articulating member is provided. The system includes a superior inferior component, a connection feature, a housing, at least one rotating member and a cutter. The superior inferior component is engaged to a structure. The connection feature is engaged with the superior inferior component. The housing is engaged with the connection feature. The rotating member is configured to rotate within the housing. The first cutter is engaged with the rotating member such that the rotation of the rotating member translates into rotation of the first cutter, wherein the first cutter rotates about an axis that is generally parallel to a natural surface of the bone.

### RELATED APPLICATIONS

This application claims the benefit of and priority to:

U.S. Provisional Patent Application No. 61/777,538 filed Mar. 12, 2013, which carries Applicants' docket no. CCF-8 PROV, and is entitled GLENOID AUGMENT PREPARATION INSTRUMENT, which is pending; and

U.S. patent application Ser. No. 13/708,799, filed Dec. 7, 2012, entitled SHOULDER ARTHROPLASTY, Attorney's docket no. CCF-3 CIP1, which is pending.

U.S. Provisional Patent Application No. 61/568,530, filed Dec. 8, 2011, entitled GLENOID VAULT FIXATION, Attorney's docket no. CCF-1 PROV, which is expired.

U.S. patent application Ser. No. 13/360,459, filed Jan. 27, 2012, entitled GLENOID VAULT FIXATION, Attorney's docket no. CCF-1, which is pending.

P.C.T. Patent Application No. PCT/US2012/024035, filed Feb. 6, 2012, entitled GLENOID VAULT FIXATION, Attorney's docket no. CCF-1 PCT, which is pending.

U.S. patent application Ser. No. 13/367,165, filed Feb. 6, 2012, entitled GLENOID VAULT FIXATION, Attorney's docket no. CCF-3, which is pending.

U.S. Provisional Patent Application No. 61/604,391, filed Feb. 28, 2012, entitled GLENOID VAULT FIXATION, Attorney's docket no. CCF-4 PROV, which is expired.

U.S. Provisional Patent Application No. 61/615,560, filed Mar. 26, 2012, entitled GLENOID VAULT FIXATION, Attorney's docket no. CCF-5 PROV, which is expired.

U.S. Provisional Patent Application No. 61/701,484, filed Sep. 14, 2012, entitled GLENOID VAULT FIXATION, Attorney's docket no. CCF-5 PROV, which is expired.

P.C.T. Patent Application No. PCT/US2012/068605, filed Dec. 7, 2012, entitled SHOULDER ARTHROPLASTY, Attorney's docket no. CCF-3 PCT, which is pending.

The above-referenced documents are hereby incorporated by reference in their entirety.

### BACKGROUND

1. Technical Field

The present disclosure relates to total shoulder arthroplasty. More specifically, but not exclusively, the present disclosure relates to systems, methods and instruments for preparing bone to receive a glenoid construct.

2. The Relevant Technology

Posterior glenoid bone deficiency may be addressed by implanting an articulating component that may have an augment portion. The surface of the glenoid may have to be prepared such that the augment portion of the articulating component appropriately interfaces with the surface of the glenoid.

Hence, there may be a need for systems, methods and instruments which may enable preparing of the glenoid for receiving an articulating component that may have an augment portion.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered of its scope.

FIG. 1A is perspective view of a superior inferior component;

FIG. 1B is a sectional view of the superior inferior component of FIG. 1;

FIG. 2A is perspective view of a connection feature;

FIG. 2B is a sectional view of the connection feature of FIG. 2A;

FIG. 3 is a sectional view of the connection feature of FIG. 2A engaged with the superior inferior component of FIG. 1A;

FIG. 4 is a perspective view of the connection feature of FIG. 2A engaged with the superior inferior component of FIG. 1A;

FIG. 5A is a perspective view of an instrument;

FIG. 5B is a sectional view of the instrument of FIG. 5A.

FIG. 6A is a perspective view of another instrument;

FIG. 6B is a sectional view of the instrument of FIG. 6A.

FIG. 7A is a perspective view of yet another instrument;

FIG. 7B is a sectional view of the instrument of FIG. 7A.

FIG. 8 is a perspective view of yet another instrument; and

FIG. 9 is a perspective view of a glenoid component.

### DETAILED DESCRIPTION

Standard medical planes of reference and descriptive terminology are employed in this specification. A sagittal plane divides a body into right and left portions. A mid-sagittal plane divides the body into equal right and left halves. A coronal plane divides a body into anterior and posterior portions. A transverse plane divides a body into superior and inferior portions. Anterior means toward the front of the body. Posterior means toward the back of the body. Superior means toward the head. Inferior means toward the feet. Medial means toward the midline of the body. Lateral means away from the midline of the body. Axial means toward a central axis of the body. Abaxial means away from a central axis of the body.

With reference to the related applications mentioned above, the present disclosure relates to instruments and methods for preparing a glenoid to receive an augmented glenoid component. These instruments may include a component that is secured to a superior-inferior trial, broach, or implant component, and is not necessarily directly secured to bone. These instruments may include a feature that allows a bone preparation instrument to be pivoted about the secured component. The feature may be included on a sleeve component. By pivoting the bone preparation instrument about the secured component, the glenoid may be prepared for complementary fit with the augmented glenoid component.

A system may be used for preparing a glenoid surface to receive a glenoid component comprising an augmented portion. Such preparation of the glenoid surface during shoulder arthoplasty and revision surgeries may provide a more secured interface between the glenoid component and the bone.

The system may include a superior inferior component, a connection feature, a housing, at least one rotating member and at least one cutter. The superior inferior component may be embedded into the scapula through the glenoid surface. The connection feature may be engaged to the superior inferior component. Further, the connection feature may be engaged to the housing or accommodation means. The housing may receive a rotating member. The rotating member may be engaged to the cutter. The rotating member may be configured to rotate about an axis. The rotation of the rotating member may translate into rotation of the cutter. The rotation of the cutter may shape the surface of the bone, thereby facilitating reception of a glenoid component comprising an augmented portion.

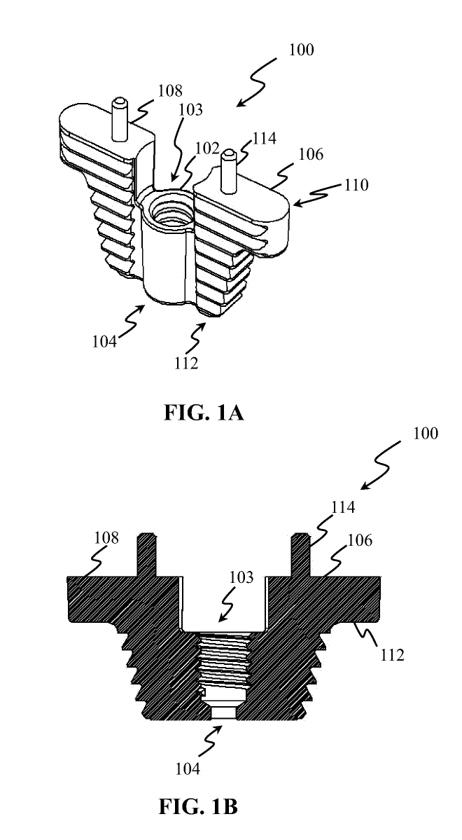

Referring to FIGS. 1A and 1B a superior inferior (SI) component 100 may be engaged to a structure. The SI component 100 may be engaged to a structure, such as a bone by embedding the SI component 100 in the bone. The SI component 100 may include a bore 103, which may be a central bore, extending at least partially through the body or central ring 102 in a longitudinal direction and may extend entirely through the central ring 102. The SI component 100 includes a distal end 104 and may include two arms 106, 108 extending from the central ring 102. The arms 106,108 may be integral to the body 102, or may be separately formed. The arms 106, 108 include a proximal end 110 and a distal end 112. Portions of the arms 106, 108 extend proximally from the central ring 102 giving the SI component 100 a V or U-shaped configuration. The extension of the arms 106 proximally may be substantially parallel and substantially the same length, wherein the arms are coplanar; however the arms may differ in length slightly as well which may give the SI component 100 a J-shape, wherein the arms are not) coplanar. The extension of the arms 106, 108 may be collinear and the arms 106, 108 may prove to be minor images if a cross section is taken of the SI component 100. The body of the SI component 100 may be longer than it is wide from a top view providing a narrow footprint when the SI component 100 sits within the bone with the arms 106, 108 narrower than the central ring 102.

Each of the arms 106,108 may include a protruding feature 114 extending from the proximal end 110. The protruding features may facilitate engagement of a connection feature with the SI component 100.

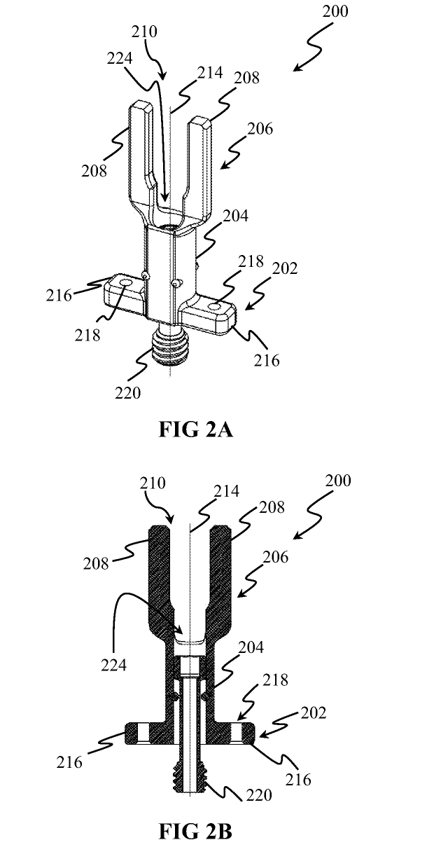

Referring to FIGS. 2A and 2B the SI component 100 may be engaged with a connection feature 200. The connection feature 200, or connecting unit or connection means may include a distal broach contacting portion 202, a medial portion 204 and a proximal forked portion 206. The proximal forked portion 206 may include at least one arm 208 or prong that extends proximally from the medial portion 204. In the example shown, the connection feature 200 includes two arms 208 that defines a central channel 210 that is located between the arms 208.

The medial portion 204 may extend distally from the proximal forked portion 206, and may be substantially cylindrical. The medial portion 204 may also be square, triangular or otherwise irregularly shaped. The medial portion 204 may intersect the broach-contacting portion 202. The broach-contacting portion 202 may include at least one lateral arm 216 that extends substantially perpendicularly to a central axis 214 that extends through the central channel 210 and further through the medial portion 204. The central axis 214 may also be referred to as a longitudinal axis.

Each of the two lateral arms 216 may include an aperture 218 shaped to receive a complimentary protruding feature 114 provided in the SI component 100.

In an alternate embodiment, the lateral arms 210 may include a male mating feature, such as a peg, and the SI component 100 may include a complimentary female mating feature, such as a groove or aperture. When the connecting feature 200 engages the SI component 100, at least a portion of the male mating feature of the SI component may be contained within the female mating feature of the lateral arm 216, thus substantially preventing rotation of the complete connection feature 200 about the central axis 214.

It may be noted that the connection feature 200 may be provided with a rotation feature enabling at least a position of the connection feature 200 to rotate. Such a feature may enable a housing engaged with the connection feature 200 to rotate about the central axis 214. The rotation enabled may be 360 degrees. Alternatively, rotation by 180 degrees may be enabled. The rotation feature may be provided in the medial portion 204. A bearing arrangement may be provide to enable rotation.

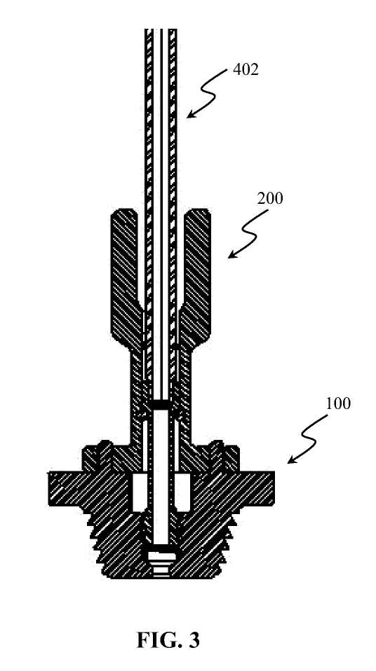

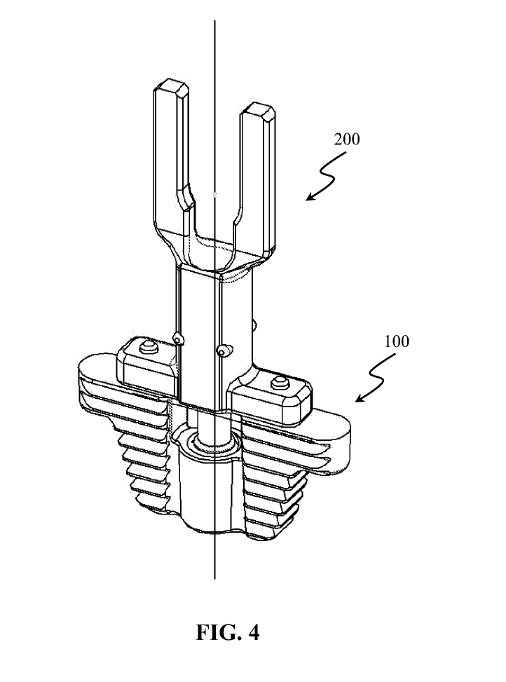

Referring to FIGS. 3 and 4 the connecting feature 200 is shown operationally coupled to the SI component 100. The connecting feature 200 may also include an SI component-fastener 220 extending distally from a substantially central location between the lateral arms 216 of the broach-contacting portion 202, and may be at least partially threaded. The fastener 220 may be shaped to be received within the central ring 102 of the SI component 100.

When the connection feature 200 is operationally engaged with the SI component 100, as seen best in FIG. 3, at least a portion of the protruding feature 114 on the SI component 100 may be received by the aperture 218 on the lateral arm 216.

An aperture 224 may be defined in the medial portion 204, such that the SI component-fastener 220 may be accessible and engageable through the aperture 224. An actuating instrument 402 may pass through the aperture 224 and engage the SI component-) fastener 220. The fastener 220 may be rotated about the longitudinal axis 214 by the actuating instrument 402 to engage the threads and move the fastener 220 distally into the SI component 100. The actuating instrument 402 only rotates the fastener 220 about the central axis or longitudinal axis 214 without rotating the lateral arms 216 of the distal broach contacting portion 202.

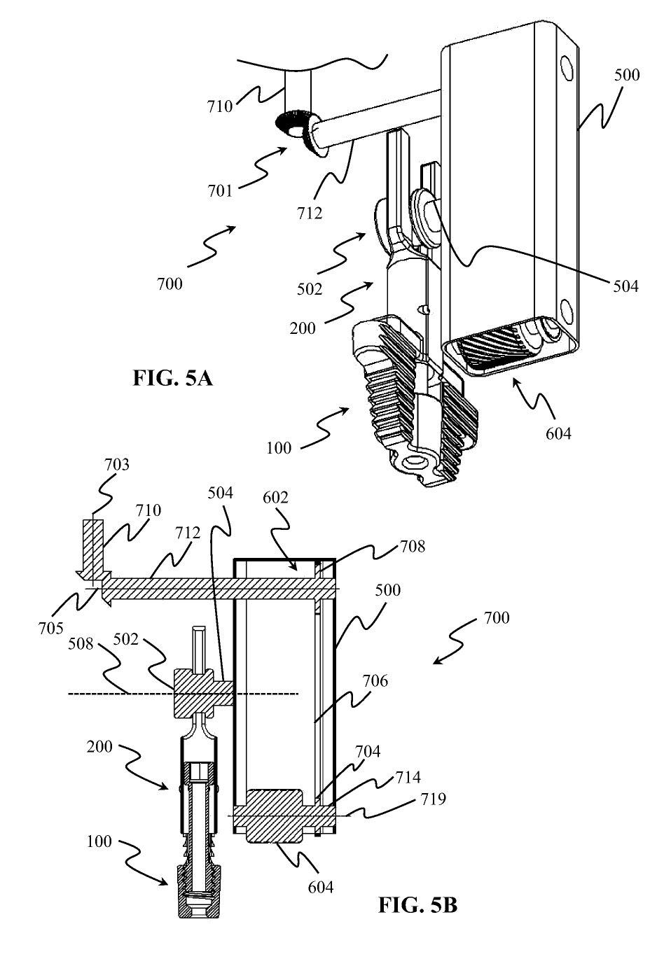

Referring to FIGS. 5A and 5B, a perspective view of a housing 500 engaged to the connection feature 200 is illustrated. The housing 500 may be engaged to the connecting feature 200 by an engagement feature 502 and a neck portion 504. The neck portion 504 may extend between the housing 500 and the engagement feature 502. The engagement feature 502 may be substantially cylindrical and may include a circumferential groove 506 or channel that extends at least partially around the engagement feature 502. The groove 506 may be shaped to engage the at least one arm 208 of the proximal forked portion 206 of the connection feature 200. The engagement feature 502 may be free to spin or pivot in its engagement with the connection feature 200, thereby enabling the housing 500 to pivot about a pivot axis 508. The pivot axis 508 may intersect the longitudinal axis 214 of the connection feature 200 at an angle. The angle of intersection may be 90 degrees.

Referring FIGS. 5A and 5B the housing 500 may support a cutting means 604 or first cutter or cutter. The housing 500 may receive at least one rotating member 602, which may rotate within the housing 500. The rotating member 602 may include a single gear or plurality of gears.

The cutting means 604 may be engaged with the rotating member 602, such that the rotation of the rotating member 602 translates into rotation of the cutting means 604. The cutting means 604 used to shape the surface of the glenoid to receive the glenoid component may be a single cutter.

In an alternate embodiment, the cutting means 604 may have plurality of cutters to shape the surface of the glenoid. The plurality of cutters may be aligned along a single axis. Alternatively, one or more cutters may be aligned along intersecting axes. Further, at least two cutters may be different diameters.

Referring to FIGS. 5A and 5B, an instrument or system 700 may be used for preparing the glenoid surface to receive a glenoid component. Instrument 700 may be engaged with the SI component 100. The SI component 100 may be embedded into the scapula through the glenoid surface. The connection feature 200 may be engaged with the SI component 100. The housing 500 in turn may be engaged to the connecting feature 200 via the engagement feature 502 and the neck portion 504.

The housing 500 may receive the rotating member 602, a second gear 704, and a transmission member 706 or transmission means. The rotating member 602, the second gear 704, and the transmission member 706 may be within the housing or accommodation means 500.

The rotating member may include a first shaft 712. A first gear 708 may be mounted over the first shaft 712. A rotation imparting member 710 may be engaged with the first shaft 712. The engagement between the rotation imparting member 710 and the first shaft may define a beveled gear transmission mechanism 701.

The rotation imparting member 710 may be configured to rotate about an axis 703, such that the rotation of the rotation imparting member 710 translates into rotation of the first shaft 712 about another axis 705. The axes 703 and 705 may intersect at an angle. The angle may be 90 degrees. Alternatively the intersection may define an oblique angle.

The transmission member 706 may engage the first gear 708 and the second gear 704. The transmission member 706 may be a gear belt or a third gear. The transmission member 706) may be disposed between the first gear 708 and the second gear 704. The transmission member 706 may rotate as the first gear 708 rotates, thereby transmitting the rotation to the second gear 704. The second gear 704 may be mounted over a second shaft 714. Further, the cutter 604 may also be mounted over the second shaft 714. The cutter 604 may be disposed between the rotating member 602 and the connection feature 200.

The axis of rotation of the first gear 708 may be parallel to the axis of rotation of the second gear 704. The rotation of the second gear 704 may translate into rotation of the second shaft 714 over which the first cutter 604 may be mounted. The first cutter 604 may rotate about an axis 719 that may be generally parallel to the natural glenoid surface. The axis 719 may be incident to an axis that is perpendicular to the pivot axis 721. The angle of incidence may be 90 degrees. Alternatively, the axes may intersect to define an oblique angle.

The rotating member 602 may be parallel to the first cutter 602 and the connection feature 200. The axis of rotation of the rotating member 602 may be angular to the axis of rotation of the first cutter 604. In an alternate embodiment, the axis of rotation of the rotating member 602 may be offset from to the axis of rotation of the first cutter 604. Further, the axis of rotation of the rotation member 702 may be parallel to the first cutter 604.

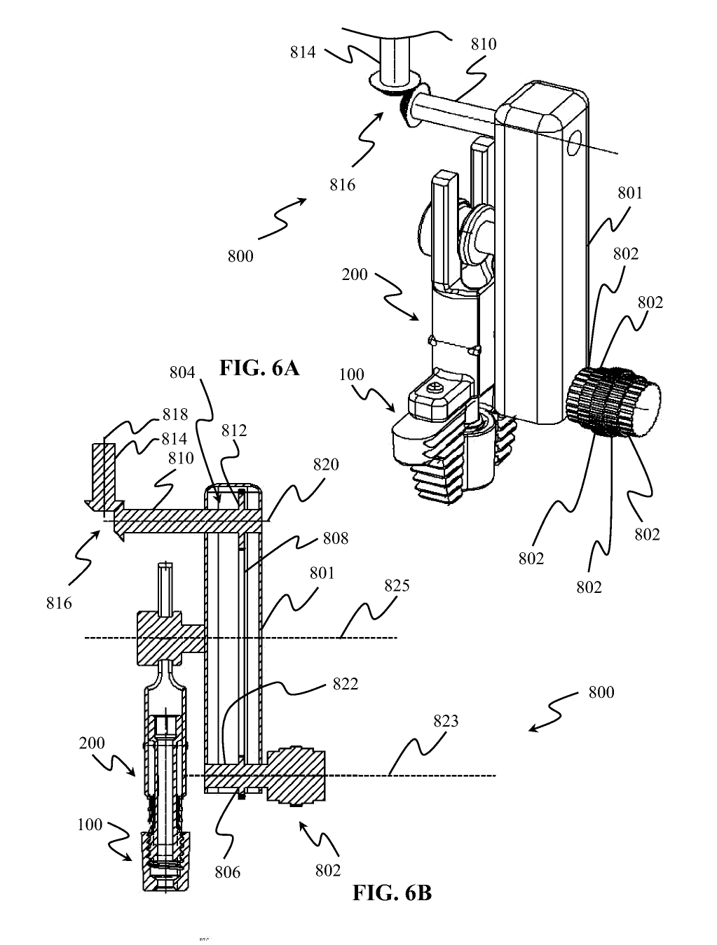

Referring to FIGS. 6A and 6B, an alternate embodiment of an instrument 800 is illustrated. A rotating member 804 may be disposed between a plurality of cutters 802 and the connection feature 200. Alternatively, instead of providing multiple cutters 802, a single cutter may be provided.

The engagement of the housing 801 with the connection feature 200 may be similar to engagement described previously.

The housing 801 may receive the rotating member 804, a second gear 806, and a transmission member 808 or transmission means. The rotating member 804, the second gear 806, and the transmission member 808 may be within the housing 801 or accommodation means.

The rotating member 804 may include a first shaft 810. A first gear 812 may be mounted over the first shaft 810. A rotation imparting member 814 may be engaged with the first shaft 810. The engagement between the rotation imparting member 814 and the first shaft 810 may define a beveled gear transmission mechanism 816.

The rotation imparting member 814 may be configured to rotate about an axis 818 such that the rotation of the rotation imparting member 814 translates into rotation of the first shaft 810 about another axis 820. The axes 818 and 820 may intersect at an angle. The angle may be 90 degrees. Alternatively the intersection may define an oblique angle.

The transmission member 808 may engage the first gear 812 and the second gear 806. The transmission member 808 may be a gear belt 811 or third gear. The transmission member 808 may rotate as the first gear 812 rotates, thereby transmitting the rotation to the second gear 806. The second gear 806 may be mounted over a second shaft 822. Further, a plurality of cutters 802 may also be mounted over the second shaft 822. The rotating member 804 may be disposed between the plurality of cutters 802 and the connection feature 200.

The axis of rotation of the first gear 812 may be parallel to the axis of rotation of the second gear 806. The plurality of cutters 802 may rotate about an axis 823 that may be generally parallel to the natural glenoid surface. The axis 823 may be incident to an axis that is perpendicular to the pivot axis 825. The angle of incidence may be 90 degrees. Alternatively, the axes may intersect to define an oblique angle.

The axis of rotation of the rotating member may be angular to the axis of rotation of the plurality of cutters. In an alternate embodiment, the axis of rotation of the rotating member 804 may be offset from to the axis of rotation of the plurality of cutters 802. Further, the axis of rotation of the rotation member 804 may be parallel to the plurality of cutters 802.

Two or more cutters 802 may differ in their dimension such as diameter, dimension and cutting profile, among others. Such variation may enable achieving a desired topography of the glenoid surface.

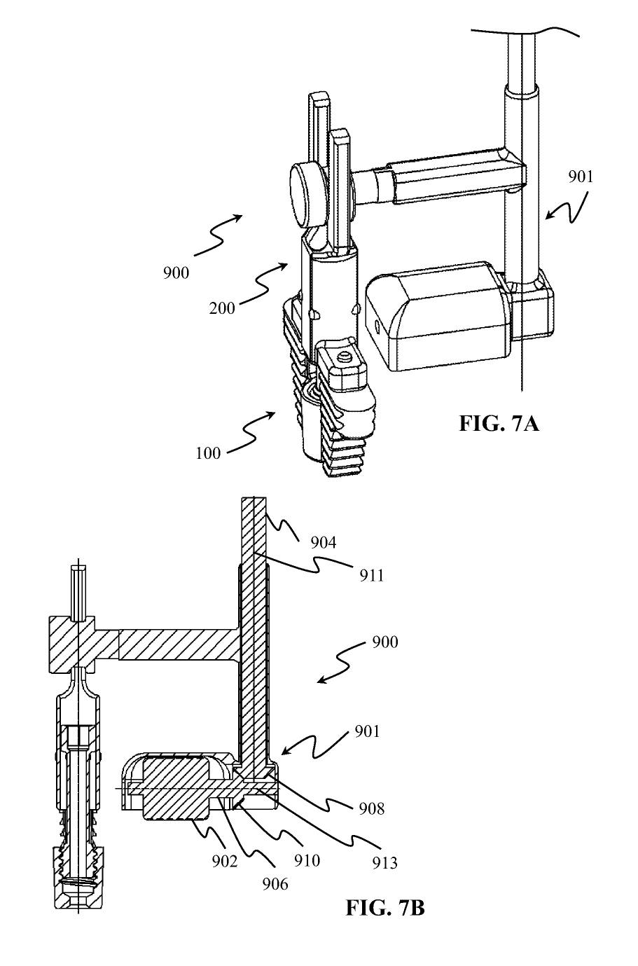

Referring FIGS. 7A and 7B, an instrument 900 is illustrated. The instrument 900 may include a cutter 902 disposed between a rotating member 904 and the connection feature 200. Instead of providing a single cutter 902, multiple cutters may be provided.

A housing 901 may be engaged with the connection feature 200 in a manner similar to the technique discussed previously.

The housing 901 may receive the rotating member 904, a shaft 906, and the cutter 902. The rotating member 904 may include a first beveled gear 908. The shaft 906 may be engaged to the cutter 902. The shaft 906 may include a second beveled gear 910 configured to interface with the first beveled gear 908. The rotation of the first beveled gear 908 along an axis 911 translates into rotation of the second beveled gear 910. The second beveled gear 910 rotates about an axis 913 that may intersect the axis 911.

A position of the cutter 902 may be disposed within the housing 901. The instant configuration may prevent dispersion of grains of the bone that are chipped off by the cutter 902. The bone grains may fly into the housing 901, thereby preventing dispersion of bone grains onto an operator.

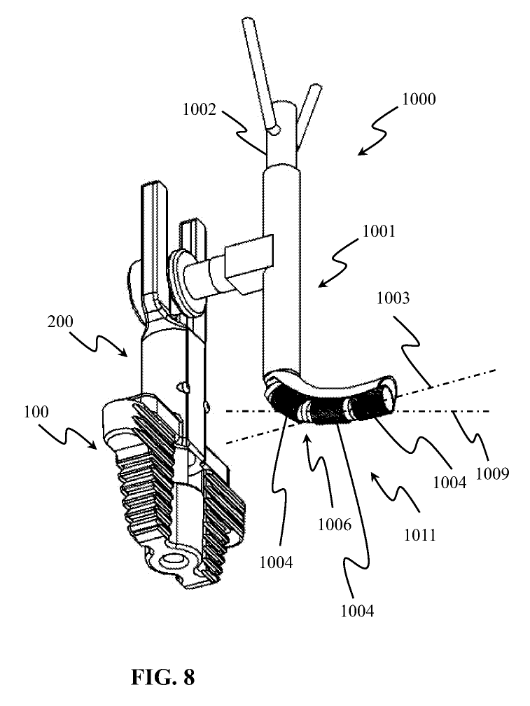

Referring to FIG. 8, an alternate embodiment of an instrument 1000 is illustrated. The housing or accommodation means 1001 may receive a rotating member 1002 configured to transfer torque to a plurality of cutters 1004. The plurality of cutters 1004 may be disposed along a curved configuration 1011. The axes 1003 and 1009 of rotation of at least two of the cutters 1004 may intersect to define an oblique angle.

A flexible means may be used for transferring torque to one or more of the cutters 1004, thereby enabling curved configuration 1011 of the cutters 1004.

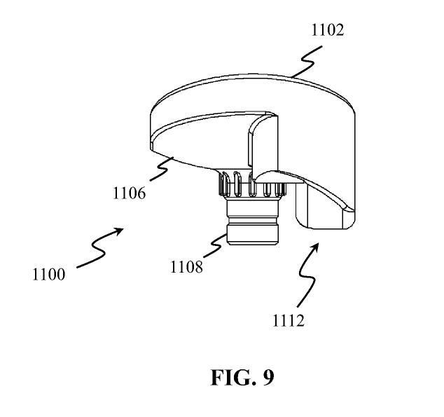

Referring to FIG. 9, a glenoid component 1100 has a curvature shaped to minor an anatomical shoulder with a semi-spherical or concave glenoid surface 1102 peripherally surrounded by a wall. The glenoid component 1100 also includes a bone-facing surface 1106 and a post 1108. The post 1108 extends from the bone-facing surface 1106 in a substantially central location of the bone-facing surface 1106. The bone facing surface 1106 may rest against the scapula. The glenoid component 1100 includes an augment 1112 extending from the bone facing surface 1106. The instruments or systems or methods of the present disclosure may be used for preparing the scapula for receiving the glenoid component 1100 that may include the augment 1112.

While these examples all include a superior-inferior component, this part may be exchanged for a superior-inferior trial component, a superior-inferior implant component, or some other glenoid base, cup, or tray unit, whether an implant, a trial or provisional component, or a broach component.

The components disclosed herein may be made from metals, polymers, ceramics, glasses, composite materials, biological materials or tissues, insulators, conductors, semiconductors, or other biocompatible or non-biocompatible materials. Different materials may be used for individual components. Different materials may be combined in a single component.

It should be understood that the present systems, kits, apparatuses, and methods are not intended to be limited to the particular forms disclosed. Rather, they are to cover all combinations, modifications, equivalents, and alternatives falling within the scope of the claims.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

In the foregoing Detailed Description, various features are grouped together in several embodiments for the purpose of streamlining the disclosure. This method of disclosure is not to) be interpreted as reflecting an intention that the embodiments of the invention require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

The present invention may be embodied in other specific forms without departing from its spirit or essential characteristics. It is appreciated that various features of the above described examples and embodiments may be mixed and matched to form a variety of other combinations and alternatives. It is also appreciated that this system should not be limited simply to systems, methods and instruments for preparing bone to receive a glenoid construct. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A system for preparing a bone for receiving an articulating member, the system comprising:
a superior inferior component configured to be engaged to a structure;
a connection feature engaged with the superior inferior component;
a housing engaged with the connection feature;
at least one rotating member configured to rotate within the housing; and
a first cutter engaged with the rotating member such that the rotation of the rotating member translates into rotation of the first cutter, wherein the first cutter rotates about an axis that is generally parallel to a natural surface of the bone.

2. The system of claim 1, wherein the axis of rotation of the rotating member is angular to the axis of rotation of the first cutter.

3. The system of claim 1, wherein,
the axis of rotation of the rotating member is parallel to the axis of rotation of the first cutter; and
the axis of rotation of the rotating member is offset from to the axis of rotation of the first cutter.

4. The system of claim 1, wherein,
the rotating member comprises a first bevel gear; and)
the first cutter is engaged to a shaft, wherein the shaft comprises a second bevel gear configured to interface with the first bevel gear.

5. The system of claim 1, wherein,
the rotating member comprises a first gear; /
the first cutter is engaged to a shaft, wherein the shaft comprises a second gear configured to interface with the first gear; and
the axis of rotation of the first gear is parallel to the axis of rotation of the second gear; and
the first gear is engaged with the second gear such that the rotation of the first gear translates into rotation of the second gear.

6. The system of claim 1, further comprising a plurality of cutters, wherein the first cutter is among the plurality of cutters, wherein axis of rotation of at least two of the cutters intersect to define an oblique angle.

7. The system of claim 1, wherein the system comprises a transmission member, wherein the transmission member interfaces with the first gear and the second gear such that the rotation of the first gear translates into rotation of the second gear.

8. The system of claim 1, further comprising a plurality of cutters, wherein at least two of the cutters have different diameters.

9. The system of claim 1, wherein the connection feature comprises a longitudinal axis, and wherein the housing is engaged with the connection feature such that the housing pivots about an axis which is incident at an angle with the longitudinal axis of the connection feature.

10. The system of claim 1, wherein the connection feature comprises a longitudinal axis, and wherein the housing is engaged with the connection feature such that the housing is operable to swing about the longitudinal axis of the connection feature.)

11. A method for preparing a bone for receiving an articulating member, the method comprising:
engaging a superior inferior component to a structure;
engaging a connection feature with the superior inferior component;
engaging an housing with the connection feature;
enabling rotation of at least one rotating member within the housing; and
transmitting the rotation of the at least one rotating member to a first cutter, such that the first cutter rotates about an axis that is generally parallel to a natural surface of the bone.

12. The method of claim 11, imparting rotation in the rotating member by a rotation imparting member, wherein an axis about which the rotation imparting member rotates is incident at an angle with the axis about which the rotating member rotates.

13. The method of claim 11, further comprising rotating a plurality of cutters, wherein the first cutter is among the plurality of cutters, about a single axis of rotation.

14. The method of claim 11, wherein enabling rotation of the rotating member comprises rotation the rotating member about an axis which is perpendicular to the axis of rotation of the first cutter.

15. The method of claim 11, wherein transmitting the rotation of the rotating member to the first cutter comprises:
engaging a flexible member with the rotating member and the first cutter; and
transmitting the rotation via the flexible member.

16. The method of claim 11, wherein engaging the housing with the connection feature comprises, enabling the housing to operably swing about a longitudinal axis of the connection feature by at least 180 degrees.

17. A system for shaping a surface of a bone, the system comprising:
an accommodation means for receiving a rotating member, wherein the accommodation means is configured to pivot about a pivot axis; and
a cutting means engaged with the rotating member such that cutting means rotates about an axis which is incident to an axis that is perpendicular to the pivot axis.

18. The system of claim 17, further comprising a connection means for engaging the accommodation means with the bone, wherein the cutting means is disposed between the rotating member and the connection means.

19. The system of claim 17, further comprising a connection means for engaging the accommodation means with the bone, wherein the rotating member is disposed between the cutting means and the connection means.

20. The system of claim 17, wherein the cutting means comprises a cutting surface, wherein the cutting surface is partially enclosed in the accommodation means.
